(19) United States
(12) Patent Application Publication
(10) Pub. No.: US 2025/0049595 A1
(43) Pub. Date: Feb. 13, 2025

(54) ZOTAROLIMUS-ROSUVASTATIN ELUTING BIODEGRADABLE STENT

(71) Applicant: Dev GADHIA, Woodstock, MD (US)

(72) Inventor: Dev GADHIA, Woodstock, MD (US)

(21) Appl. No.: 18/233,183

(22) Filed: Aug. 11, 2023

(51) Int. Cl. A61F 2/90; A61F 2/92; A61L 31/02; A61L 31/04; A61L 31/16

(52) U.S. Cl. CPC A61F 2/90; A61F 2/92; A61L 31/022; A61L 31/04; A61L 31/16; A61F 2210/00

(57) ABSTRACT

A drug-eluting stent for insertion in a blood vessel includes a tubular mesh structure composed of a magnesium alloy and a poly-L-lactide layer that is micro arc oxidized disposed over the tubular mesh structure. The biodegradable drug-eluting stent further includes Zotarolimus and Rosuvastatin integrated into the poly-L-lactide layer to provide controlled and sustained drug release. Advantageously, wherein the drug-eluting stent is configured to be deployed in the blood vessel to provide drug delivery to a target site.

### TECHNICAL FIELD

In at least one aspect, the present invention is related to drug-eluting stents.

### BACKGROUND

Patients that suffer from high levels of cholesterol, a condition known as hyperlipidemia, and simultaneously need percutaneous coronary intervention can benefit from drug-eluting stents. However, it has been shown only about a twenty (20) percent success rate for achieving the target LDL cholesterol level when utilizing current drug-eluting stent technology.

Accordingly, there is a need for improved drug-eluting stent technology for treating coronary artery disease.

### SUMMARY

In at least one aspect, a biodegradable drug-eluting stent for insertion in a blood vessel is provided. The biodegradable drug-eluting stent includes a tubular mesh structure composed of a magnesium alloy and a poly-L-lactide layer disposed over the tubular mesh structure. The biodegradable drug-eluting stent further includes Zotarolimus and Rosuvastatin integrated into the poly-L-lactide layer to provide controlled and sustained drug release. Advantageously, wherein the drug-eluting stent is configured to be deployed in the blood vessel to provide drug delivery to a target site.

In another aspect, the biodegradable drug-eluting stent can potentially help underdeveloped regions reduce expenses for treating coronary artery disease and its complications because the stent is cost-effective.

In another aspect, the biodegradable drug-eluting stent addresses plaque buildup, artery constriction, and stent biodegradability.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Side view of a biodegradable drug-eluting stent.

FIG. 1B. Cross-sectional view of a biodegradable drug-eluting stent of FIG. 1A.

FIG. 1C. Schematic illustrating placement of the biodegradable drug-eluting stent of FIGS. 1A and 1B in a blood vessel.

FIG. 2. Schematic flowchart showing the placement of the biodegradable drug-eluting stent of FIGS. 1A and 1B in a blood vessel.

### DETAILED DESCRIPTION

Reference will now be made in detail to presently preferred compositions, embodiments and methods of the present invention, which constitute the best modes of practicing the invention presently known to the inventor. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.) and L- is a counter anion (e.g., Cl—, Br—, tosylate, etc.); single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.); percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

The compounds set forth herein (e.g., Zotarolimus and Rosuvastatin) may be used per se or as pharmaceutically acceptable derivatives. The latter term includes salts, esters, and other derivatives generally considered acceptable by pharmaceutical standards. Useful derivatives, for example, include salts of organic and inorganic acids such as sulfates, phosphates, hydrohalide salts, carboxylate salts, etc., as well as esters of carboxylic acid or hydroxyl substituents, ethers of hydroxyl substituents, amides of amino substituents, as well as carbamates, ureas, etc. Synthesis of these derivatives is conventional, and well known to those skilled in pharmaceutical chemistry. For example, compounds bearing hydroxyl groups may be converted to esters by customary techniques of organic chemistry, such as reaction with an acyl halide, carboxylic acid anhydride, or by esterification with an acid while removing byproduct water. In some cases, derivation may be desired to facilitate compounding of the pharmaceutical into an acceptable form such as tablets, powder, aqueous dispersion, capsule, etc., or may be useful in assisting bioavailability of the drug following administration, for example, by rendering the compound more or less soluble. In many cases, such as, for example, esters, ureas, carbamates, ethers, etc., the derivative may function as “prodrug,” which liberates the active form by biological transformation, i.e., by enzymatic hydrolysis of an ester functionality, as is well known to the pharmaceutical chemist. It should also be appreciated that may have one or more hydrogen atoms (H) replaced by deuterium (D). In particular, OH groups can be replaced by OD groups (e.g., acids and alcohols).

Throughout this application, where publications are referenced, the disclosures of these publications in their entirety are hereby incorporated by reference into this application to more fully describe the state of the art to which this invention pertains.

### Abbreviations

“DES” means drug-eluting stent.
“LDL” means low-density lipoprotein.

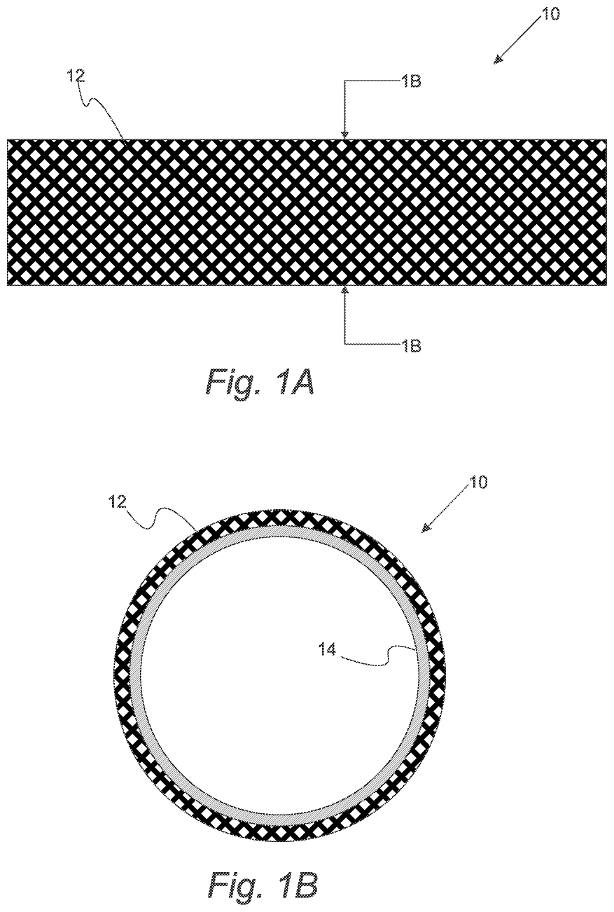

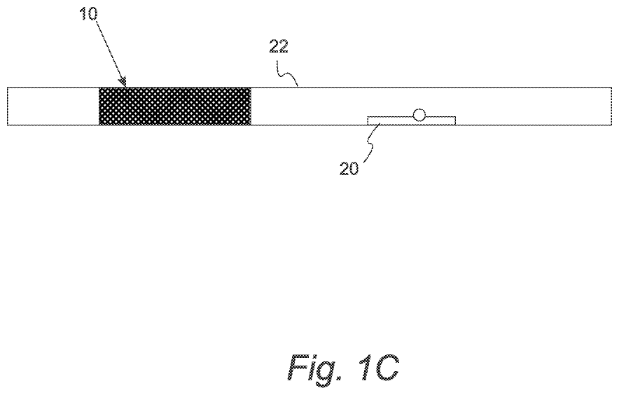

Referring to FIGS. 1A and 1 B, schematics of a drug-eluting stent for insertion in a blood vessel are provided. Drug-eluting stent 10 includes a tubular mesh structure 12 composed of a magnesium alloy and a poly-L-lactide layer 14 disposed over the mesh structure 12. Characteristically, Zotarolimus and Rosuvastatin or pharmaceutically derivatives thereof are integrated into poly-L-lactide layer 14 to provide drug delivery to a target site 20. Drug-eluting stent 10 provides controlled and sustained drug release (of Zotarolimus and Rosuvastatin). Moreover, drug-eluting stent 10 is configured to be deployed in the blood vessel 22. Such a target site 22 can be a plaque which typically includes fatty substances, cholesterol, cellular waste products, calcium, and other substances. Advantageously, the magnesium alloy and a poly-L-lactide layer 14 are biodegradable. However, the magnesium alloy (e.g., WE43 alloy) is sufficiently long lasting to be useful. For example, the magnesium alloy can have a degradation rate of greater than about 1% per year (1.35% per year for WE43 alloy).

Zotarolimus reduces the risk of late stent thrombosis (blood clotting 1-3 years after stent implantation) and reduces artery blockage. Moreover, Zotarolimus is one of the most effective drugs at reducing repeated percutaneous coronary intervention. Zotarolimus is described by chemical formula 1 (see, pubchem.ncbi.nlm.nih.gov/compound/Zotarolimus; the entire disclosure of which is hereby incorporated by reference):

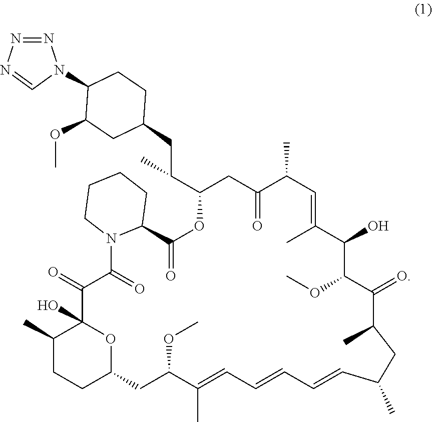 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Rosuvastatin is a type of statin which is known to reduce LDL cholesterol. Rosuvastatin is characterized by 19 elimination half-lives. The typical daily dose is between 5 and 40 mg which targets a plasma concentration of 0.5 ng/ml. Moreover, when Rosuvastatin is dispersed along another medium (MAO/PLLA polymer), its contents still are effective out of tablet form. Advantageously, Rosuvastatin reduces risk of Acute Stent Thrombosis. Rosuvastatin can be used in the stent in extended release form to maximize drug's absorption. Rosuvastatin is described by the chemical formula 2 (see, pubchem.ncbi.nlm.nih.gov/compound/Rosuvastatin; the entire disclosure of which is incorporated by reference):

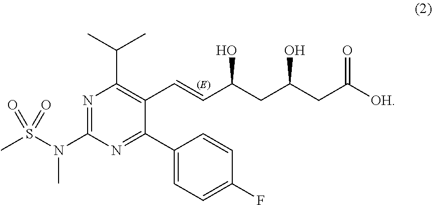 [SMILES: CC(C)c1nc(N(C)S(C)(=O)=O)nc(-c2ccc(F)cc2)c1/C=C/[C@@H](O)C[C@@H](O)CC(=O)O]

In another aspect, the Zotarolimus and Rosuvastatin are dispersed in the poly-L-lactide layer. In a refinement, the poly-L-lactide layer is a micro-arc oxidized.

In another aspect, the mesh structure 12 is a metallic, expandable, porous scaffold. In a refinement, the metallic, expandable, porous scaffold includes chromium to increase elasticity and flexibility.

In another aspect, the magnesium alloy includes magnesium, yttrium, and rare earth elements. The rare earth elements can include neodymium, zirconium, and gadolinium. In a refinement, the magnesium alloy includes magnesium in an amount from about 93 to 94 weight percent magnesium and yttrium in an amount of about 4 to 5 weight percent of the total weight of the magnesium alloy. In a further refinement, the magnesium alloy includes the rare earth elements in an amount from about 2 to 3 weight percent of the total weight of the magnesium alloy. In a refinement, the magnesium alloy includes calcium to increase plasticity and strength. In a further refinement, the magnesium alloy includes calcium in an amount of 0.2 to 1.0 weight percent of the total weight of the magnesium alloy. In still another refinement, the magnesium alloy further includes manganese to assist in dispersing the Zotarolimus and Rosuvastatin.

In another aspect, tubular mesh structure 12 has a diameter from about 2.25 mm to 4.0 mm and a length from about 8 mm to 40 mm. In a further refinement, the tubular mesh structure includes struts having a thickness from about 50 micrometers to 150 micrometers.

In another aspect, poly-L-lactide layer 14 with the Zotarolimus and Rosuvastatin integrated therein can be formed by a number of techniques known to those skilled in the art. Examples of such techniques include but are not limited to spray coating and dip coating from solutions that include Zotarolimus and Rosuvastatin. Moreover, electrophoretic deposition can be used to attach charged particles to the stent thereby forming a drug release layer.

In another aspect, the loading of Zotarolimus and Rosuvastatin or pharmaceutically derivatives thereof are each independently from about 50 μg/mm² to 200 μg/mm². The loading is expressed as micrograms of each drug per square millimeters of the stent surface area.

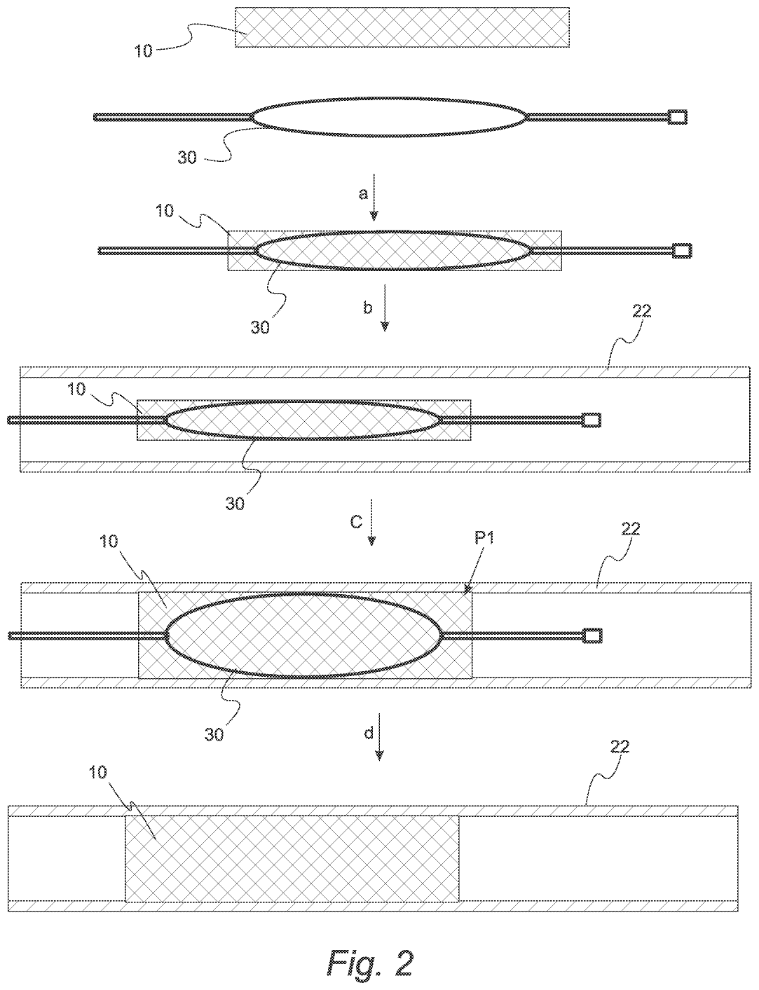

Referring to FIG. 2, a schematic flowchart depicting the insertion of the drug-eluting stent into a blood vessel is provided. In step a), a balloon catheter 30 is placed in the center opening of the drug-eluting stent 10 described above. In step b), the stent is placed in a blood vessel 20 as seen in 1C. In step c), after the drug-eluting stent 10 is positioned at a predefined position P1, the balloon catheter 30 is inflated such that the stent expands to contact the inner blood vessel wall. In step d), the balloon catheter 30 is deflated and removed leaving the stent attached to the blood vessel wall.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A drug-eluting stent for insertion in a blood vessel, comprising:
a) a tubular mesh structure composed of a magnesium alloy;
b) a poly-L-lactide layer disposed over the tubular mesh structure; and
c) Zotarolimus and Rosuvastatin or pharmaceutically derivatives thereof integrated into the poly-L-lactide layer to provide controlled and sustained drug release, wherein the drug-eluting stent is configured to be deployed in the blood vessel to provide drug delivery to a target site.

2. The drug-eluting stent of claim 1, wherein the Zotarolimus and Rosuvastatin are dispersed in the poly-L-lactide layer.

3. The drug-eluting stent of claim 1, wherein the poly-L-lactide layer is a micro-arc oxidized.

4. The drug-eluting stent of claim 1, wherein the tubular mesh structure includes struts having a thickness from about 50 micrometers to 150 micrometers.

5. The drug-eluting stent of claim 1, wherein the tubular mesh structure is a metallic, expandable, porous scaffold.

6. The drug-eluting stent of claim 5, wherein the metallic, expandable, porous scaffold includes chromium.

7. The drug-eluting stent of claim 1, wherein magnesium alloy includes magnesium, yttrium, and rare earth elements.

8. The drug-eluting stent of claim 7, wherein the rare earth elements include neodymium, zirconium, and gadolinium.

9. The drug-eluting stent of claim 7, wherein the magnesium alloy includes magnesium in an amount from about 93 to 94 weight percent magnesium and yttrium in an amount of about 4 to 5 weight percent of the total weight of the magnesium alloy.

10. The drug-eluting stent of claim 7, wherein the magnesium alloy includes the rare earth elements in an amount from about 2 to 3 weight percent of the total weight of the magnesium alloy.

11. The drug-eluting stent of claim 7, wherein the magnesium alloy further includes calcium to increase plasticity and strength.

12. The drug-eluting stent of claim 7, wherein the magnesium alloy further includes manganese to assist in dispersing the Zotarolimus and Rosuvastatin.

13. The drug-eluting stent of claim 1, wherein the tubular mesh structure has a diameter from about 2.25 mm to 4.0 mm and a length from about 8 mm to 40 mm.

14. The drug-eluting stent of claim 1, wherein the loading of the Zotarolimus and Rosuvastatin on the stent are each independently from about 50 μg/mm² to 200 μg/mm².
